(19) United States
(12) Patent Application Publication
(10) Pub. No.: US 2015/0141605 A1
(43) Pub. Date: May 21, 2015

(54) PRODUCTION OF POLYPROPYLENE FROM RENEWABLE RESOURCES

(71) Applicant: David Bradin, Chapel Hill, NC (US)

(72) Inventor: David Bradin, Chapel Hill, NC (US)

(21) Appl. No.: 14/398,553

(22) PCT Filed: May 3, 2013
(86) PCT No.: PCT/US13/39439

§ 371 Date: Nov. 3, 2014

Related U.S. Application Data
(60) Provisional application No. 61/641,936, filed on May 3, 2012.

(51) Int. Cl. C08F 10/06; C08F 22/02; C08F 10/08; C07C 5/333; C07C 2/08; C07C 5/25; C07C 6/04; C08F 24/00; C07C 1/22

(52) U.S. Cl. CPC C08F 10/06; C08F 24/00; C08F 22/02; C08F 10/08; C07C 1/22; C07C 2/08; C07C 5/2556; C07C 6/04; C07C 5/333; C07C 2523/755; C07C 2523/42; C07C 2523/44; C07C 2523/30
USPC 526/268; 526/318.25; 526/348; 526/348.6; 526/351; 585/329; 585/240; 585/241; 585/242

(57) ABSTRACT

Processes for forming propylene from methanol are disclosed. The processes involve converting methanol to a product mixture comprising ethylene and propylene, separating the ethylene from the propylene, dimerizing a first portion of the ethylene to form a product mixture comprising 1-butylene, isomerizing the 1-butylene to form a mixture of cis and trans 2-butylene, and performing olefin metathesis on a second portion of the ethylene and the mixture of cis and trans 2-butylene. In one embodiment, the methanol is produced by converting syngas to methanol, and in one aspect of this embodiment, the syngas, or a portion thereof, is produced from renewable feedstocks. In this aspect, renewable propylene is produced. The propylene can be polymerized to form polypropylene or co- or terpolymers thereof, and when the propylene is made from renewable resources, the resulting polymer is a renewable polymer.

This application claims priority of U.S. provisional application No. 61/641,936 filed on May 3, 2012. All applications are included herein in its entirety by reference.

### FIELD OF THE INVENTION

The present invention relates to the production of propylene, and polypropylene, from renewable resources.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

Most of the world's polyolefins, such as polyethylene, polypropylene, and co-polymers thereof, are derived from crude oil. It would be desirable to provide a source of polyolefins from biomass, particularly if the polyolefins can be obtained at a cost approximating that of conventional polyolefin synthesis. The present invention provides such a source of polyolefins, as well as the olefinic starting materials.

### SUMMARY OF THE INVENTION

A process for preparing propylene from methanol is disclosed. The methanol can be produced from a variety of feedstocks, including biomass, landfill waste, coal, natural gas, and a variety of other materials. When the methanol is produced from biomass, the process can be used to produce a propylene product with a Carbon 14 signature showing that it is derived from materials other than crude oil, i.e., is a “green” propylene.

The propylene produced by this process can be polymerized, either by itself, or with other olefinic materials, to produce polypropylene, and copolymers, terpolymers, and other such polymers thereof. Where the propylene is derived from renewable materials, the resulting polymer is a “green” polymer.

In one embodiment, the chemistry involves converting methanol to olefins, which are primarily ethylene and propylene, but which can include a minor amount of butylene. Ideally, the ethylene and propylene are separated by distillation. A portion of the ethylene is then dimerized to form butylene. Where the dimerization produces 1-butylene, the butylene can be isomerized to form 2-butylenes (i.e., cis and trans 2-butylene). The cis and trans isomers are both capable of participating in olefin metathesis with ethylene to form a product mixture containing propylene. The propylene can be obtained by distillation, and any remaining ethylene and butylene recycled and reused, for example, in the olefin metathesis step of the process.

The polyolefins are prepared by converting feedstocks, such as biomass or other renewable materials, or, alternatively, coal, methane or other suitable feedstocks, to syngas, converting syngas to methanol, converting methanol to C₂₋₄ olefins, and isolating ethylene, propylene and butylene from the C₂₋₄ olefin-rich stream (optionally in the presence of the related paraffins, which can optionally be removed at a later time). When the feedstocks are renewable, the process can produce “green” monomers that can be polymerized to form “green” polymers.

In one embodiment, residual ethylene and/or butylene can be co-polymerized with the propylene to yield co-polymers and ter-polymers.

As there is an abundant supply of biomass, and other renewable feedstocks which can be converted to syngas, the chemistry described herein can produce a substantial volume of olefins and/or polyolefins that are not derived from petroleum or petroleum-based products.

The olefins described herein can be combined with olefins derived from conventional sources, if desired.

The olefins can be derived, in whole or in part, by methanol synthesis using syngas formed using, for example, coal, glycerol, ethanol, methanol, methane, lignin, cellulose, hemicellulose, black liquor, or biomass (including corn stover, switchgrass, bagasse, sawdust, recycled paper, and the like) as a starting material. The methane can be present as part of a renewable feedstock, for example, as the product from anaerobic digestors, landfill gas, and the like.

The olefin metathesis can be run at high yields, and adds significantly to the total weight of the propylene (or polypropylene) that is obtained.

Thus, polyolefin products that are typically obtained solely from petroleum can be obtained in significant yields from methanol, using relatively inexpensive catalysts, and the methanol can be obtained using biomass and/or other renewable resources as feedstocks.

In one embodiment, the methanol-to-olefins chemistry produces a minor amount of paraffins, which can be separated from the olefins by distillation, dehydrogenated to form additional olefins, separated from the polymers after a mixture of olefins and paraffins is sent to a polymerization reactor. The separation of C₂₋₄ alkanes is significantly easier following polymerization than from the corresponding C₂₋₄ olefins.

The resulting olefins and/or polymers can help reduce U.S. dependence on foreign crude oil, and, when derived from renewable feedstocks, have environmental benefits, such as a reduction in greenhouse gases.

### DEFINITIONS

The terms “about” and “essentially” mean ±10 percent.

The term “comprising” is not intended to limit inventions to only claiming the present invention with such comprising language. Any invention using the term comprising could be separated into one or more claims using “consisting” or “consisting of” claim language and is so intended.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

### DETAILED DESCRIPTION

A process for producing propylene is disclosed. The process involves subjecting methanol to methanol-to-olefin synthesis to form a reaction mixture primarily including ethylene and propylene, and separating the ethylene from the propylene. A portion of the ethylene is subjected to olefin dimerization to form butylenes, and where 1-butylene is formed, it can be isomerized to form 2-butylenes (cis and trans isomers, both of which can participate in olefin metathesis reactions). Ethylene and 2-butylenes can be subjected to olefin metathesis to form a product mixture rich in propylene, which propylene can then be isolated by distillation.

In one embodiment, the methanol used to produce the initial olefin mixture is derived in whole or in part from renewable materials. For example, lignocellulosic materials such as trees, grasses, and the like, can be gasified to produce syngas. Syngas can be converted, optionally following syngas cleanup and water-gas shift chemistry to optimize the ratio of carbon monoxide and hydrogen, to methanol.

The propylene produced by the process can be blended with propylene derived from petroleum-based sources, if desired, or used by itself to produce a polypropylene. Polypropylene produced by this process is readily distinguishable from polypropylene produced from crude oil, in that the Carbon 14 footprint is completely different. Thus, the product produced from this process is different than conventional polypropylene, and identifiable as a bio-based material.

Further, the propylene can be combined with other monomers, such as ethylene, butylene, styrene, butadiene, acrylonitrile, acrolein, (methyl)acrylate, (methyl)methacrylate, maleic acid, malic acid, maleic anhydride and the like to produce desired co- and ter-polymer chemical building blocks.

Certain embodiments of the present invention will now be further described in more detail, in a manner that enables the claimed invention so that a person of ordinary skill in this art can make and use the present invention.

Unless otherwise indicated, all numbers expressing reaction conditions, stoichiometries, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon the specific analytical technique. Any numerical value inherently contains certain errors necessarily resulting from the standard deviation found in its respective testing measurements.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs. If a definition set forth in this section is contrary to or otherwise inconsistent with a definition set forth in patents, published patent applications, and other publications that are herein incorporated by reference, the definition set forth in this specification prevails over the definition that is incorporated herein by reference.

In some embodiments, the processes described herein are integrated processes. As used herein, the term “integrated process” refers to a process which involves a sequence of steps, some of which may be parallel to other steps in the process, but which are interrelated or somehow dependent upon either earlier or later steps in the total process.

The following definitions will further define the invention:

The term “alkyl”, as used herein, unless otherwise specified, refers to a saturated straight, branched, or cyclic hydrocarbon of C₁₋₆, and specifically includes methyl, ethyl, propyl, isopropyl, butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl.

The term “olefin” refers to an unsaturated straight, branched or cyclic hydrocarbon of C₂₋₁₀, and specifically includes ethylene, propylene, butylenes (1-butene and 2-butene), isobutylene, pentene, cyclopentene, isopentene, hexene, cyclohexene, 3-methylpentene, 2,2-dimethylbutene, 2,3-dimethylbutene, 1-heptene, 2-heptene, 3-heptene, 1-octene, 2-octene, 3-octene, 4-octene, 1-nonene, 2-nonene, 3-nonene, 4-nonene, 1-decene, 2-decene, 3-decene, 4-decene, and 5-decene.

### I. Raw Materials

The methanol used in methanol-to-olefin synthesis can be derived from any known process. In one embodiment, the process does not start with syngas generation and subsequent conversion of syngas to methanol, but rather, starts with methanol produced at a separate facility. That is, methanol can be obtained as a feedstock, and the methanol converted to olefins, which are subsequently converted to propylene. However, in one embodiment, the methanol is produced, via methanol synthesis, from syngas.

The raw materials used in the methanol synthesis can include any material that can be converted to syngas, including both renewable and non-renewable materials. Examples of non-renewable materials include coal and methane (natural gas), though bio-based equivalents are known. In one embodiment, biomass is converted to char, and the char converted to syngas. In this embodiment, the char is similar to coal, in that it is essentially elemental carbon. Methane is known as “natural gas,” because it forms in nature, often by action of methanogenic bacteria. As such, landfills, farms such as dairy farms, and other sources of bio-based materials can be subjected to conditions that produce methane, frequently in combination with carbon dioxide. The methane can be converted to syngas. In one aspect of this embodiment, methane is subjected to steam reforming to produce syngas with a 3/1 ratio of hydrogen to carbon dioxide. Methane synthesis only uses two equivalents of hydrogen for each carbon dioxide, so there is an extra equivalent of hydrogen. This can be reacted with the carbon dioxide in a process known as the “reverse water gas shift” to produce extra syngas. Thus, there are true equivalents to coal and natural gas, in addition to conventional biomass to syngas chemistries.

In another embodiment, the raw materials to produce syngas are either municipal solid waste (“MSW”), or “refuse-derived fuel,” (“RFD”) where MSW is treated to remove glass and metal, leaving behind materials that can be gasified. The MSW and/or RDF can be used in combination with landfill gas.

In another embodiment, the raw materials for producing syngas are biomass, including cellulosic biomass, such as corn stover, bagasse, switchgrass, algae, wood, sawdust, or waste streams derived from biomass, including the crude glycerol from biodiesel synthesis and hemicellulose, lignin or black liquor derived from cellulose and/or paper production.

Representative carbon-containing feedstocks include timber harvesting residues, softwood chips, hardwood chips, tree branches, tree stumps, leaves, bark, sawdust, paper pulp, corn stover, wheat straw, rice straw, sugarcane bagasse, switchgrass, miscanthus, animal manure, municipal solid waste, municipal sewage, commercial waste, used tires, grape pumice, almond shells, pecan shells, coconut shells, coffee grounds, grass pellets, hay pellets, wood pellets, cardboard, paper, plastic, rubber, cloth, coal, lignite, coke, lignin, and/or petroleum. Mixtures of any of these feedstocks can be used.

In this embodiment, biomass and waste streams can be converted to useful fuel and other products, rather than being sent to a landfill or, in the case of black liquor, often dumped into water supplies.

In this embodiment, the process can be compatible with cellulosic ethanol production. That is, cellulosic ethanol will require the separation of cellulose from lignin and, optionally, hemicellulose. Delignification generates black liquor, which can be converted to syngas, and, ultimately, methanol. The hemicellulose can be depolymerized and, often inefficiently, fermented to alcohol, or also used as a feedstock to produce methanol as described herein.

### II. Methanol Production

Methanol production from synthesis gas (“syngas”) is relatively well known. A brief discussion is provided below.

### Synthesis Gas (Syngas) Production

Syngas can be produced by any known means, such as by one or more of gasification, pyrolysis, devolatilization, steam reforming, and partial oxidation of one or more feedstocks recited herein.

It is known in the art to convert a variety of feedstocks, such as coal, methane, methanol, ethanol, glycerol, biomass such as corn stover, switchgrass, sugar cane bagasse, sawdust, and the like, black liquor, and lignin to synthesis gas (see, for example, [http://www.biocap.ca/files/biodiesel/dalai.pdf]). The water-gas-shift reaction plays an important role in the conversion of certain of these feedstocks to hydrogen via steam gasification and pyrolysis. Catalytic steam gasification can give high yields of syngas at relatively low temperatures.

Biomass can be converted to syngas using a variety of known methods, including thermal gasification, thermal pyrolysis and steam reforming, and/or hydrogasification, each of which can produce syngas yields of 70-75% or more.

The syngas-generation unit or step may be a gasifier, such as a fluidized-bed gasifier. In variations, the gasifier type may be entrained-flow slagging, entrained flow non-slagging, transport, bubbling fluidized bed, circulating fluidized bed, or fixed bed. Some embodiments employ known gasification catalysts. “Gasification” and “devolatilization” generally refer herein to the reactive generation of a mixture of at least CO, CO₂, and H₂, using oxygen, air, and/or steam as the oxidant(s).

In some embodiments, syngas is produced by the methods taught in U.S. patent application Ser. No. 12/166,167, entitled “METHODS AND APPARATUS FOR PRODUCING SYNGAS,” filed Jul. 1, 2008.

Gasification is known as the art to convert a variety of feedstocks, such as coal, methane, methanol, ethanol, glycerol, biomass such as corn stover, switchgrass, sugar cane bagasse, sawdust, and the like, black liquor, municipal solid waste, and lignin to synthesis gas. Many gasifiers have been developed, and one exemplary gasifier is that sold by TCG Energy (www.tcgenergy.com). Another gasifier of noteable mention is the Thermoselect (www.iwtonline.com). Also, see, for example, [http://www.biocap.ca/files/biodiesel/dalai.pdf]. The Wiley gasifier, covered by one or more of U.S. Pat. Nos. 7,638,070; 7,857,995; 7,968,006; 8,017,040; 8,017,041 and 8,021,577 can also be used.

The water-gas-shift reaction plays an important role in the conversion of certain of these feedstocks to hydrogen via steam gasification and pyrolysis. Catalytic steam gasification can give high yields of syngas at relatively low temperatures. Biomass can be converted to syngas using a variety of known methods, including thermal gasification, thermal pyrolysis and steam reforming, and/or hydrogasification each of which can produce syngas yields of 70-75% or more.

These approaches are well known in the art, and need not be described in more detail. Gasification of coal was used in England and in the United States to produce “town gas” to light the city streets over a hundred years ago. The German war effort was fueled by coal gasification during World War II. Currently over 40% of South African motor fuel is derived from coal gasification, as well as 100% of their aviation fuel. Major suppliers of gasification technology include Sasol, Lurgi, GE, Conoco-Phillips, and Shell with a significant number of plants operating worldwide.

### Syngas Cleanup

Syngas is converted to methanol by contact with a suitable catalyst under reactive conditions. Depending on the quality of the syngas, it may be desirable to purify the syngas prior to the methanol-generating reactor to remove carbon dioxide produced during the syngas reaction, and any sulfur compounds, if they have not already been removed. This can be accomplished by contacting the syngas with a mildly alkaline solution (e.g., aqueous potassium carbonate) in a packed column. This process can also be used to remove carbon dioxide from the product stream.

One example of a suitable syngas cleanup process is called extractive Merox. Merox is an acronym for mercaptan oxidation. It is a proprietary catalytic chemical process developed by UOP, and used in oil refineries and natural gas processing plants to remove mercaptans from a variety of products, including syngas, LPG, propane, butanes, light naphthas, kerosene and jet fuel by converting them to liquid hydrocarbon disulfides.

The Merox process requires an alkaline environment which, in some of the process versions, is provided by an aqueous solution of sodium hydroxide (NaOH), a strong base, commonly referred to as caustic. In other versions of the process, the alkalinity is provided by ammonia, which is a weak base.

The overall chemical reaction can be generalized as follows:

4RSH+O₂→2RSSR+2H₂O

The catalyst in some versions of the process is a water-soluble liquid, such as methanol. In other versions, the catalyst is impregnated onto charcoal granules.

The Merox process is usually more economical than using a catalytic hydrodesulfurization process for much the same purpose, though the latter can be used if desired. In addition, the Selexol (UOP) and Rectisol (Lurgi) processes are commonly used for acid gas removal.

### Water-Gas Shift Chemistry

Biomass gasification typically produces a synthesis gas with an approximately 1/1 ratio of hydrogen to carbon monoxide. It is generally desirable to have a 2/1 ratio of hydrogen to carbon monoxide when carrying out methanol synthesis. Accordingly, it is important to increase the amount of hydrogen in the syngas. This is typically done using water-gas shift chemistry, which involves reacting water with carbon monoxide to produce hydrogen and carbon dioxide. The general reaction is shown below:

CO+H₂O→CO₂+H₂

The resulting syngas can be used in methanol synthesis, optionally after being subjected to syngas cleanup to remove various impurities (including nitrogen and sulfur impurities). Further, where the ratio of hydrogen to carbon monoxide is less than 2/1, the syngas is ideally subjected to water-gas shift conditions to produce syngas at or near this ratio.

### Methanol Synthesis

In 1923, the German chemists Alwin Mittasch and Mathias Pier developed a means to convert synthesis gas into methanol. This technology is described in U.S. Pat. No. 1,569,775, and the process used a chromium and manganese oxide catalyst, and required extremely vigorous conditions—pressures ranging from 50 to 220 atm, and temperatures up to 450° C.

Modern methanol production is more efficient through the use of catalysts (commonly copper-zinc) capable of operating at lower pressures. The modern low pressure methanol (LPM) process was developed by ICI in the late 1960s with the technology now owned by Johnson Matthey.

The carbon monoxide and hydrogen react on a catalyst to produce methanol. Today, the most widely used catalyst is a mixture of copper, zinc oxide, and alumina first used by ICI in 1966. At 5-10 MPa (50-100 atm) and 250° C., it can catalyze the production of methanol from carbon monoxide and hydrogen with high selectivity (>99%):

CO+2H₂→CH₃OH

Representative catalysts and conditions are described, for example, in Vanden Bussche and Froment, 1996 “A steady-state kinetic model for methanol synthesis and the water gas shift reaction on a commercial Cu/ZnO/Al₂O₃ catalyst,” Journal of Catalysis, 161, pp. 1-10.

It is worth noting that the production of synthesis gas from methane produces three moles of hydrogen gas for every mole of carbon monoxide, while the methanol synthesis consumes only two moles of hydrogen gas per mole of carbon monoxide. One way of dealing with the excess hydrogen is to inject carbon dioxide into the methanol synthesis reactor, where it, too, reacts to form methanol according to the equation:

CO₂+3H₂→CH₃OH+H₂O

Mature technologies available for biomass gasification are being used for methanol production. For instance, woody biomass can be gasified to water gas (a hydrogen-rich syngas), by introducing a blast of steam in a blast furnace. The water-gas/syngas can then be converted to methanol using standard methods. The net process is carbon neutral, since the CO₂ byproduct is required to produce biomass via photosynthesis. The overall chemical process is summarized below:

2C₁₆H₂₃O₁₁+19H₂O+O₂→42 H₂+21CO+11CO₂→21CH₃OH+11CO₂

This process is extremely advantageous, in that 21 of the 32 carbon atoms present in the starting material are converted to methanol, and only 11 of the 32 carbon atoms are converted to carbon dioxide. The carbon dioxide can optionally be captured, and sequestered, used in the food industry, used for enhanced oil extraction, and the like.

### III. Methanol-to-Olefin/Methanol-to-Propylene Chemistry

Methanol can be passed over a methanol-to-olefin or methanol-to-propylene catalyst to generate a mixture of ethylene and propylene, optionally with a minor amount of butylene and higher olefins. Any suitable methanol-to-olefin or methanol-to-propylene catalyst can be employed. That is, any material exhibiting activity for converting methanol to one or more olefins can be employed, though it can be preferred to use materials that are selective for propylene over ethylene.

In some embodiments, the methanol-to-olefin catalyst comprises an aluminosilicate zeolite, such as one selected from the group consisting of ZSM-5, ZSM11, ZSM-12, ZSM-23, ZSM-35, and ZSM-48. ZSM catalysts tend to be preferred for maximizing propylene yield, with H-ZSM5 and modified H-ZSM5 catalysts being particularly preferred.

In some embodiments, the methanol-to-olefin catalyst comprises a silicoaluminophosphate (“SAPO”), such as a SAPO selected from the group consisting of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, and SAPO-56.

SAPOs can be synthesized by forming a mixture containing sources of silicon, aluminum, and phosphorus mixed with an organic template, and then crystallizing the molecular sieve at reaction conditions. Many factors affect the form the molecular sieve takes, including the relative amounts of the different components, the order of mixing, the reaction conditions (e.g. temperature and pressure) and the choice of organic template.

A preferred methanol-to-olefin catalyst that can be used is SAPO-34 (also referred to herein as H-SAPO-34). The preparation of SAPO-34 is known in the art, as exemplified in U.S. Pat. No. 4,440,871, issued to Union Carbide on Apr. 3, 1984, which is incorporated by reference herein in its entirety. SAPO-34 has a three-dimensional microporous crystal framework structure and an empirical composition on an anhydrous basis of (SiₓAl_{y}P_{z})O₂ where x, y and z represent the mole fractions of silicon, aluminum, and phosphorus, respectively, and where typically x+y+z=1.

Without being limited by any hypothesis, it is believed that the framework structure can trap organic intermediates (such as ethylbenzenes) deriving from methanol.

These organic intermediates act as organic reaction centers that catalyze the olefin-forming reactions in cooperation with active sites over the surface of the catalyst. Olefins, such as ethylene and propylene, are small enough to exit the micropores of SAPO-34.

SAPO-34 offers a good combination of catalyst activity, selectivity, and durability. Ethylene/propylene ratios in H-SAPO-34 may be driven by increased temperatures; at higher temperatures ethylene selectivities increase. At higher temperatures, coking rates are higher so more frequent regeneration is typically needed.

It can be further beneficial for the methanol-to-olefin catalyst to employ silicoaluminophosphates that also include at least one transition metal. Preferably, the transition metal is selected from manganese, nickel, or cobalt. The process of incorporating the transition metal may be accomplished through any one of the standard methods well known to those skilled in the art. In one embodiment, a solution of the desired metal is first made by dissolving the desired amount of the metal-containing compound in water under mild conditions. The temperature of mixing is dependent upon the solubility of the metal compound in water, or another medium.

The amount of metal which is incorporated may vary over a wide range depending, at least in part, on the selected silicoaluminophosphate (or other material) and on the incorporation method. The amount of metal incorporated is measured on an atomic metal basis in terms of silicon-to-metal ratio. The silicon-to-metal atomic ratios are in the range from about 0.1:1 to about 1000:1, preferably from about 1:1 to about 500:1, and most preferably from about 10:1 to about 200:1.

In some embodiments wherein nickel is the selected transition metal, high methanol-to-olefin conversion can be accomplished by using Ni-SAPO-34. For example, use of Ni-SAPO-34 is described in Inui and Kang, “Reliable procedure for the synthesis of Ni-SAPO-34 as a highly selective catalyst for methanol to ethylene conversion,” Applied Catalysis A: General, vol. 164, 211-223, 1997. As taught therein, ethylene selectivity is 88% over Ni-SAPO-34, at 425-450° C. and close to atmospheric pressure.

In some embodiments, ethylene is the preferred olefin. In other embodiments, propylene is the preferred olefin. In some embodiments, it is preferred to produce higher quantities of C₃₊ olefins, including propylene, butenes (e.g., 1-butene and 2-butene), and optionally higher olefins. Generally, process conditions and catalysts can be selected to optimize selectivity to one particular olefin, which can be ethylene or propylene in particular embodiments. Or, process conditions and catalysts can be selected to optimize selectivity to total olefins rather than non-olefins (e.g., alkanes, aromatics, and CO₂). Process conditions and catalysts can also be selected to maximize methanol conversion, maximize yield of total olefins, maximize yield of C₂-C₃ olefins, or maximize yield of a specific olefin such as ethylene or propylene.

Preferably, methanol is completely or nearly completely converted in the olefin-forming process step. In various embodiments, selectivities to ethylene are in the 50-75 mol % range, while selectivities to propylene are in the 25-50 mol % range. In preferred embodiments, negligible quantities of methane and carbon dioxide are produced during olefin formation. Production of carbon dioxide can occur, however, in the gas phase away from catalyst surfaces, or possibly catalyzed by other non-selective surfaces present, such as walls of the reactor.

The olefin-forming reaction is exothermic. The catalyst can produce coke, and if that occurs, the catalyst can be periodically regenerated by hot air or oxygen. A plurality of reactors can be employed, so that when one is being regenerated, the other reactors can continue operation.

The temperature for the olefin-forming step(s) can be 375-425° C., for example. Higher temperatures will generally lead to higher selectivity to ethylene relative to propylene, but the choice of catalyst will also dictate product distribution. Any pressure can be employed, and selection of pressure will typically be dictated by economics and integration with an overall process. Reactor configurations are further discussed below.

The process of converting methanol into propylene can, in one embodiment, involve two separate reactions. The first reaction, which can occur in a pre-reactor, partially converts methanol into dimethyl-ether (DME) and water on an aluminum oxide catalyst. In the main reactor, dimethyl-ether and unconverted methanol mixture from the DME reactor are converted on zeolite-based catalyst with a high selectivity toward low-molecular-weight olefins ranging from C₂ to C₈, with the peak for propylene. The intermediate formation of dimethyl ether is described, for example, in U.S. Pat. No. 8,415,518 and U.S. Pat. No. 7,045,672. In particular, U.S. Pat. No. 7,045,672 discloses processes for making olefin product from an oxygenate feed that includes a step of pretreating a fresh or regenerated metalloaluminophosphate molecular sieve, which is low in carbon content, with a dimethyl ether composition. The dimethyl ether in the composition forms a hydrocarbon co-catalyst within the pore structure of the molecular sieve, and the pretreated molecular sieve containing the co-catalyst is used to convert oxygenate to an olefin product, with high selectivity to light olefins. The MTPROP® catalyst from Clariant, for example, converts methanol to propylene in the presence of steam. The highly selective catalyst favors the formation of propylene.

The conversion conditions can be carefully chosen to favor the production of propylene over other olefins. The reaction temperatures are typically in the range of from about 350° C. to about 600° C. The lower portion of this temperature range with certain catalysts is known to favor the production of propylene, and the upper portion typically favors the production of ethylene at the expense of propylene. The methanol to olefin and methanol to propylene reactions are carried out in the presence of a catalyst, which is ideally a molecular sieve catalyst. Suitable catalysts include zeolitic molecular sieves in the calcined form, including those represented by the general formula:

Me₂/nO:Al₂O₃:xSiO₂:yH₂O

where Me is a cation, x is the framework SiO₂ to Al₂O₃ ratio and has a value from about 2 to infinity, n is the cation valence and y has a value of about 2 to 100 or more and more typically about 2 to 25.

Zeolites which may be used include chabazite—also referred to as Zeolite D, clinoptilolite, erionite, ferrierite, mordenite, Zeolite A, Zeolite P, ZSM-5, ZSM-11, and MCM-22. Zeolites having a high silica content (i.e., those having framework silica to alumina ratios greater than 100 and typically greater than 150 with good results achieved at a silica to alumina mole ratio of about 150:1 to 800:1) are especially preferred. One such high-silica-content zeolite having the structure of ZSM-5 is silicalite, as the term used herein includes both the silicapolymorph disclosed in U.S. Pat. No. 4,061,724 and also the F-silicate disclosed in U.S. Pat. No. 4,073,865. Best results are obtained with ZSM-5 or ZSM-11 or a mixture thereof. Such catalysts are sometimes referred to as having a “pentasil-type” structure.

Suitable non-zeolitic molecular sieves are embraced by an empirical chemical composition, on an anhydrous basis, expressed by the empirical formula:

(EL_{X}Al_{y}P_{z})O₂

where EL is an element selected from the group consisting of silicon, magnesium, zinc, iron, cobalt, nickel, manganese, chromium and mixtures thereof, x is the mole fraction of EL and is at least 0.005, y is the mole fraction of aluminum and is at least 0.01, z is the mole fraction of phosphorous and is at least 0.01 and x+y+z=1. When EL is a mixture of metals, x represents the total amount of the element mixture present. Preferred elements (EL) are silicon, magnesium and cobalt with silicon being especially preferred.

Blends of zeolitic-type catalysts, blends of non-zeolitic molecular sieve catalysts and blends of both zeolitic-type and non-zeolitic-type molecular sieve catalysts can be used.

After the methanol-to-olefins chemistry has been carried out, ideally producing a mixture including predominantly ethylene and propylene, and, more ideally, producing predominantly propylene, the ethylene and propylene can be separated, for example, by distillation.

Dimerizing Ethylene to 1-butylene

Ethylene can be dimerized by any catalytic process that is known in the art for converting ethylene into light alpha-olefins. Clearly, preference is given to catalysts that can produce a high dimer selectivity (1-butylene).

It is possible to use a certain number of catalytic zirconium oligomerization systems, but they essentially produce mixtures of dimers, trimers and tetramers.

Such catalytic systems have been described in the following documents, for example: FR-B-2 669 921, which describes the use of a system obtained by pre-reacting a mixture of alkyl zirconate and ether with an aluminum compound; FR-B-2 689 500, which describes the use of a catalytic zirconium system resulting from reacting an oxo carboxylate zirconium complex with an aluminum compound. The reaction can be carried out in the presence of a ligand such as a carboxylic acid, an ester, an anhydride, an amine, a nitrile or an amide; FR-B-2 693 455, which more particularly describes a catalytic zirconium system resulting from mixing: zirconium compound with general formula:

ZrXₓY_{y}O_{z}

(in which X=Cl or Br;Y =—OR, —N(R)₂ or —OCOR; R=a hydrocarbyl radical containing 1 to 30 carbon atoms; x and y=0-4; z=0 or 0.5; and x+y+2z=4); with an organic compound with formula R₁O—CR′₁R′₂—OR₂ (in which R′₁ and R′₂ =H or a hydrocarbyl radical containing 1 to 30 carbon atoms; R₁ and R₂=a hydrocarbyl radical containing 1 to 30 carbon atoms); then with a halogenohydrocarbylaluminum with formula AIR″ₙX₃₋₁ (in which R″=a hydrocarbyl radical containing 1 to 6 carbon atoms; X=Cl or Br; and n=1 or 2); FR-B-2 748 018, which describes the oligomerizing ethylene using a catalytic zirconium solution obtained by mixing a zirconium compound with an organic compound selected from acetals and ketals, esters, ketones, ethers, amines, nitriles, anhydrides, acid chlorides, amides, aldehydes, thioethers, sulphides and alkyl bisulphides, thiophenes, thioureas and phosphines, and a chlorinated or brominated hydrocarbylaluminum. An additive comprising a quaternary ammonium salt can be added to this catalytic solution to reduce undesirable deposits of solid polymer on the walls of the reactor and the tubes of the heat exchangers.

To increase the activity of such catalytic compositions, as described in FR-B-2 759 922, for example, it is possible to add a tris(hydrocarbyl) aluminum compound with formula AlR₃ (in which R₃ is a hydrocarbyl radical containing 1 to 6 carbon atoms).

However, to obtain sufficient selectivities for dimers (1-butylene), such catalytic systems have to be used at relatively high temperatures and pressures (for example temperatures of the order of 130° C. and pressures of the order of 9 MPa). Even in this case, the butylenes have to be separated from hexenes and octenes by fractionation.

Further, in the dimerization step of the process of the invention, it is preferable to use catalytic systems that allow better selectivity for butylenes, such as those used in commercial “Alphabutol®” processes.

In a process of that type, the catalyst used may consist of a solution based on alkyl titanate resulting, for example, from the interaction between an alkyl titanate and an aluminum compound with formula AlR₃ or AlR₂H in which R represents a hydrocarbyl radical (the aluminum compound is, for example, a trialkylaluminum or hydrogenodialkylaluminum compound).

FR-B-2 552 079 describes a process for synthesizing 1-butylene by dimerizing ethylene in the presence of a catalyst constituted (a) by a preformed mixture of an alkyl titanate and an ether, in particular in an ether/alkyl titanate mole ratio of 0.5:1 to 10:1, and (b) an aluminum compound with formula AlR₃ (trialkylaluminum) or AlR₂H (hydrogenodialkylaluminum). The ether is preferably selected from diethyl ether, diisopropyl ether, dibutyl ether, methyl tertiobutyl ether, tetrahydrofuran, 1,4-dioxan, dihydropyran and ethylene glycol dimethyl ether. Preferably, the catalytic system results from the interaction in a hydrocarbon medium of a trialkylaluminum with a near stoichiometric mixture of an alkyl titanate and tetrahydrofuran. The temperature employed to convert the ethylene to 1-butylene is 35° C. to 70° C. (more particularly about 50° C.), the pressure being 1 to 3 MPa, for example.

To improve the purity of 1-butylene as described, for example, in FR-B-2 581 381, the crude dimerization product can be mixed with an amine and distilled at a temperature above the dimerization temperature to separate better purity 1-butylene from the distillation residue containing the catalyst residues. For a dimerization temperature of 20° C. to 80° C., for example, the distillation temperature may be 90° C. to 180° C. The formula for the amine is HNR₁R₂ (in which R₁ and R₂ are each a hydrogen atom or a hydrocarbon radical and can together form an alkylene group, but are not both a hydrogen atom), for example. The amine preferably contains 5 to 22 carbon atoms.

To reduce undesirable deposits of solid polymer on the walls of the reactor and the tubes of the heat exchanger, an additive consisting a quaternary ammonium salt (as described, for example, in FR-B-2 748 019) or a polyethylene glycol or a polyethylene glycol derivative (as described, for example, in FR-B-2 748 020) can be used.

It is also possible to use a homogeneous catalyst comprising a nickel compound containing a phosphorus-containing ligand such as a phosphine or a phosphite.

It is thus possible to use a homogeneous nickel catalyst comprising a divalent nickel complex containing, for example, two molecules of tertiary phosphine (optionally associated with a divalent nickel compound containing neither water nor phosphine) and an alkylaluminum halide as described, for example, in European patent EP-B-0 646 413. The dimerization reaction can then be carried out at a pressure of about 5 MPa and at a temperature of about 40° C. The catalyst is optionally employed in an ionic liquid in a non aqueous medium with an ionic nature resulting from bringing at least one aluminum halide or alkylaluminum halide into contact with at least one quaternary ammonium halide and/or at least one quaternary phosphonium halide.

The dimerization reaction can then be carried out, for example at a pressure of 2 to 9 MPa and at a temperature of −10° C. to +60° C.

It is also possible to use a catalytic composition comprising at least one nickel compound in an ionic medium mixed or complexed with at least one tertiary phosphine carrying a functional group or a phosphite carrying a functional group as described, for example, in FR-A-2 804 622.

The dimerization catalyst is generally deactivated by an inhibitor. The reaction effluent, free of catalyst, and a concentrated deactivated catalytic solution may be recovered.

To this end, the operation is as follows: (a) an inhibited catalyst containing the reaction effluent is introduced into a flash column in which the pressure is reduced by 0.1 to 2 MPa, forming a first gas phase containing 1-butylene and ethylene and a first liquid phase containing the inhibited catalyst; (b) the first liquid phase is passed into at least one vaporization zone in which it is vaporized at a pressure that is substantially equal to that of step (a) and at a temperature which can separate a second gas phase containing 1-butylene and ethylene and a second liquid phase containing the inhibited catalyst; (c) the second liquid phase from (b) is passed through at least one thin film evaporation zone and evaporated at a pressure substantially equal to that in step (b) and under conditions that allow separation of a third gas phase containing 1-butylene, ethylene and other hydrocarbons and a concentrated deactivated catalytic solution; and (d) the first, second and third gas phases are recovered to obtain a gaseous reaction effluent which is largely free of catalyst.

That deactivation mode has been described in FR-B-2 733 497, for example.

It is also possible to operate on the ethylene feed in at least two successive steps with catalysts of different types, at least one oligomerization step being carried out in the presence of a catalytic element in a liquid-liquid two-phase medium containing an ionic medium which is not (or is only slightly) miscible with the organic phase. The second step is particularly selective for dimers. The pollutants in the apparatus can be tightly controlled since they are partially eliminated in the first step. Better use of catalyst in the two-phase medium can reduce loss of catalyst. Said operative mode has been described, for example, in FR-A-2 765 573.

Hydroisomerizing 1-butylene to 2-butylenes

To carry out hydroisomerizing 1-butylene to 2-butylenes, any process described in the prior art can be employed.

The catalytic composition used in the step for isomerizing 1-butylene to 2-butylenes can comprise a compound of a transition metal from group VIII, more particularly palladium or nickel.

In particular, the catalysts described in the following documents can be employed.

French patent FR-B-2 355 792 describes the use of a palladium catalyst (containing 0.5% by weight of palladium, for example) on an alumina support, the process comprising a pre-treatment consisting, for example, of bringing it into contact with 2% by weight of water for 2 hours under 25 atmospheres of hydrogen and at an hourly space velocity (HSV) of 40 liters per liter of catalyst per hour.

As described in FR-B-2 718 597, for example, palladium catalysts can be treated in advance with a solution of a sulfur compound then activated in a neutral or reducing atmosphere at a temperature of 20° C. to 300° C. at a pressure of 0.1 to 5 MPa and with an HSV of 50 to 600 h⁻¹. The catalyst, containing 0.05% to 10% by weight of sulfur, for example, is then brought into contact with the feed and hydrogen at a temperature of 20° C. to 200° C., at a pressure of 0.1 to 5 MPa and at an HSV of 0.5 to 10 h⁻¹ and at an H₂/olefin mole ratio of 0.01 to 1.

As described in FR-B-2 735 467, for example, the catalytic composition comprising a compound of a transition metal from group VIII may also contain a quaternary ammonium and/or phosphonium salt (molten salt). The use of a molten salt (quaternary ammonium and/or phosphonium salt) in combination with a transition metal compound, allows the isomerization reaction to be carried out at relatively low temperatures, in a closed, semi-closed or continuous system. The products are easy to separate from the polar catalyst and the latter is recycled in part to the isomerization reactor.

More particularly, with catalysts of that type, the hydroisomerization conditions may, for example, be as follows: a pressure of 2 to 2.5 MPa; a temperature of 60° C. to 120° C.; and an hourly space velocity of 3 to 6 h⁻¹ (for example 4 h⁻¹).

### V. Olefin Metathesis

As used herein, the terms “molecular redistribution” and olefin metathesis are used to refer to a process in which a mixture of olefins with a relatively wide size distribution is converted into an olefin stream with a relatively narrow size distribution. The terms “molecular averaging” and “disproportionation” are also used.

In some embodiments, the individual olefins in the methanol-to-olefins product stream are isolated and used, and there is no need to produce additional propylene from this stream. However, in other embodiments, it may be desired to maximize propylene production. This can be accomplished by isolating propylene from the initial olefin product stream, and converting the ethylene and 2-butylene in the stream to a propylene-rich stream via olefin metathesis. Olefin metathesis is well known, and representative conditions are described herein, as well as in U.S. Pat. No. 6,369,286 to Dennis O'Rear, the contents of which are hereby incorporated by reference.

Because products in the desired range are produced when the reactants have molecular weights closer to the target molecular weight, and because ethylene and 2-butylenes are very close to the target molecular weight (propylene), yields of propylene are very high. Also, following fractional distillation and isolation of the propylene, the ethylene and butylenes can be isolated and re-subjected to molecular averaging conditions.

In one embodiment of the process described herein, any paraffins produced during the methanol-to-olefin synthesis, or any portion thereof, can be dehydrogenated and combined with the olefins, before, during, or after the initial olefin metathesis step. That is, the combined olefins/paraffins can be distilled into fractions including ethylene/ethane, propylene/propane, and butylene/butane, and the propylene isolated therefrom. The ethylene can be separated from the ethane, and the butylene from the butane, or the combined alkane/alkene compositions can be subjected to molecular averaging (after the 1-butylene is subjected to isomerization to form 2-butylenes). If a dehydrogenation catalyst is present, the alkanes can form alkenes, and then form propylene, during the olefin metathesis reaction. Alternatively, all or part of the paraffin fraction, or components thereof, can be separately subjected to dehydrogenation, product isolation, and olefin metathesis.

### Catalysts for Molecular Redistribution/Averaging

A typical dehydrogenation/hydrogenation catalyst includes a platinum component and a typical metathesis catalyst includes a tungsten component. Examples of suitable catalysts are described in U.S. Pat. No. 3,856,876, the entire disclosure of which is herein incorporated by reference. The individual steps in the overall molecular averaging reaction are discussed in detail below.

### Dehydrogenation

When it is desired to dehydrogenate C₂₋₄ alkanes, or just ethane and/or butane, during the olefin metathesis step, the catalyst used must have dehydrogenation activity. Platinum and palladium or the compounds thereof are preferred for inclusion in the dehydrogenation/hydrogenation component, with platinum or a compound thereof being especially preferred. As noted previously, when referring to a particular metal in this disclosure as being useful in the present invention, the metal may be present as elemental metal or as a compound of the metal. As discussed above, reference to a particular metal in this disclosure is not intended to limit the invention to any particular form of the metal unless the specific name of the compound is given, as in the examples in which specific compounds are named as being used in the preparations.

The dehydrogenation step can be conducted by passing the linear paraffin feed over a dehydrogenation catalyst under dehydrogenating reaction conditions. The dehydrogenation is typically conducted in the presence of hydrogen, and, correspondingly, a certain percentage of oxygenates, e.g., linear alcohols, will be hydrogenated to the corresponding paraffins and then dehydrogenated to the corresponding internal olefins. Thus, the linear hydrocarbon feed may contain a substantial amount of linear oxygenates (i.e., C₂₋₄ alcohols). On a mole percent basis, this may be up to about 50 mol. % linear oxygenates although it is preferably less than 30 mol. %. On a weight percent basis of oxygen, this will generally be much less, because the linear hydrocarbons are typically made up of only one or two oxygen atoms per molecule. In this embodiment, it may be possible to recycle alcohols that might be present in the water fraction produced during Fischer-Tropsch synthesis, and thus form additional propylene.

In order to reduce or eliminate the amount of diolefins produced (such as butadiene) or other undesired by-products, the reaction conversion to internal olefins should preferably not exceed 50% and more preferably should not exceed 30% based on the linear hydrocarbon content of the feed. Preferably, the minimum conversion should be at least 15 wt. % and more preferably at least 20 wt. %.

Because of the low dehydrogenation conversions, feedstocks with a higher proportion of linear hydrocarbons having carbon atom numbers in the upper range of the desired normal alpha olefin (NAO) products (i.e., C₄) are preferred to facilitate separation of the desired NAO's based on boiling point differences between the NAO and unreacted paraffins.

The dehydrogenation is typically conducted at temperatures between about 500° F. and 1000° F. (260° C. and 538° C.), preferably between about 600° F. and 800° F. (316° C. and 427° C.). The pressures are preferably between about 0.1 and 10 atms, more preferably between about 0.5 and 4 atms absolute pressure (about 0.5 to 4 bars). The LHSV (liquid hourly space velocity) is preferably between about 1 and 50 h⁻¹, preferably between about 20 and 40 h⁻¹.

Since longer chained paraffins are more easily dehydrogenated than shorter chained paraffins, more rigorous conditions, e.g., higher temperatures and/or lower space velocities, within these ranges are typically used where shorter chain paraffins (i.e., the C₂₋₄ paraffins described herein) are dehydrogenated. The dehydrogenation is also typically conducted in the presence of a gaseous diluent, typically and preferably hydrogen. Although hydrogen is the preferred diluent, other art-recognized diluents may also be used, either individually or in admixture with hydrogen or each other, such as steam, methane, ethane, carbon dioxide, and the like. Hydrogen is preferred because it serves the dual-function of not only lowering the partial pressure of the dehydrogenatable hydrocarbon, but also of suppressing the formation of hydrogen-deficient, carbonaceous deposits on the catalytic composite. Hydrogen is typically used in amounts sufficient to insure a hydrogen to hydrocarbon feed mole ratio of about from 2:1 to 40:1, preferably in the range of about from 5:1 to 20:1.

Suitable dehydrogenation catalysts which can be used include Group VIII noble metals, e.g., iron, cobalt, nickel, palladium, platinum, rhodium, ruthenium, osmium, and iridium, preferably on an oxide support.

Less desirably, combinations of Group VIII non-noble and Group VIB metals or their oxides, e.g., chromium oxide, may also be used. Suitable catalyst supports include, for example, silica, silicalite, zeolites, molecular sieves, activated carbon alumina, silica-alumina, silica-magnesia, silica-thoria, silicaberylia, silica-titania, silica-aluminum-thora, silica-alumina-zirconia kaolin clays, montmorillonite clays and the like. In general, platinum on alumina or silicalite afford very good results in this reaction. Typically, the catalyst contains about from 0.01 to 5 wt. %, preferably 0.1 to 1 wt. % of the dehydrogenation metal (e.g., platinum). Combination metal catalysts, such as those described in U.S. Pat. Nos. 4,013,733; 4,101,593 and 4,148,833, the contents of which are hereby incorporated by reference in their entirety, can also be used.

Preferably, hydrogen and any light gases, such as water vapor formed by the hydrogenation of oxygenates, are removed from the reaction product prior to olefin metathesis, for example, by using one or more vapor/liquid separators. Since dehydrogenation produces a net gain in hydrogen, the hydrogen may be taken off for other plant uses (i.e., added to the syngas used in the Fischer-Tropsch synthesis) or as is typically the case, where the dehydrogenation is conducted in the presence of hydrogen, a portion of the recovered hydrogen can be recycled back to the dehydrogenation reactor. Further information regarding dehydrogenation and dehydrogenation catalysts can, for example, be found in U.S. Pat. Nos. 4,046,715; 4,101,593; and 4,124,649, the contents of which are hereby incorporated by reference in their entirety. A variety of commercial processes also incorporate dehydrogenation processes, in their overall process scheme, which dehydrogenation processes may also be used in the present process to dehydrogen the paraffinic hydrocarbons. Examples of such processes include the dehydrogenation process portion of the Pacol process for manufacturing linear alkylbenzenes, described in Vora et al., Chemistry and Industry, 187-191 (1990); Schulz R. C. et al., Second World Conference on Detergents, Montreaux, Switzerland (October 1986); and Vora et al., Second World Surfactants Congress, Paris France (May 1988), hereby incorporated by reference in their entirety.

### Olefin Metathesis

To carry out metathesis of 2-butylenes and ethylene, any process which has been described in the prior art can be employed, in particular process such as those commercially known as “OCT” and “Meta-4®”.

The ethylene and 2-butylenes, which optionally include, but preferably do not include propylene, are metathesized to form a desired propylene fraction. This involves using an appropriate olefin metathesis catalyst under conditions selected to convert a significant portion of the ethylene and 2-butylenes to propylene. Because this is an equilibrium reaction, the presence of propylene can be disfavored, so propylene concentrations less than about 25%, more preferably less than about 20%, still more preferably less than about 15% of the mixture of olefins are desired.

The olefins can be used directly in the olefin metathesis reaction, whereas any paraffins that might be present must be converted into olefins, in a process known as dehydrogenation or unsaturation, as described above, before they can participate in the reaction. The resulting olefins can be combined with the ethylene and 2-butylenes and the reaction mixture then subjected to olefin metathesis conditions.

Various catalysts are known to catalyze the olefin metathesis reaction. The catalyst mass used in the olefin metathesis reaction must have olefin metathesis activity. Olefin metathesis typically uses conventional catalysts, such as W/SiO₂ (or inexpensive variations). Usually, the olefin metathesis catalyst will include one or more of a metal or the compound of a metal from Group VIB or Group VIIB of the Periodic Table of the Elements, which include chromium, manganese, molybdenum, rhenium and tungsten. Preferred for inclusion in the olefin metathesis component are molybdenum, rhenium, tungsten, and the compounds thereof. Particularly preferred for use in the olefin metathesis component is tungsten or a compound thereof. As discussed, the metals described above may be present as elemental metals or as compounds of the metals, such as, for example, as an oxide of the metal. It is also understood that the metals may be present on the catalyst component either alone or in combination with other metals.

The chemistry does not require using hydrogen gas, and therefore does not require relatively expensive recycle gas compressors. The chemistry is typically performed at pressures in the range of 100-5000 psig. The chemistry is typically thermoneutral and, therefore, there is no need for additional equipment to control the temperature.

Depending on the nature of the catalysts, olefin metathesis (and dehydrogenation) may be sensitive to impurities in the feedstock, such as sulfur- and nitrogen-containing compounds and moisture, and these must be removed prior to the reaction.

The presence of excess hydrogen in the olefin metathesis zone can affect the equilibrium of the olefin metathesis reaction and to deactivate the catalyst.

Since the composition of the fractions may vary, some routine experimentation will be necessary to identify any contaminants that are present and identify the optimal processing scheme and catalyst to use in carrying out the invention.

The process conditions selected for carrying out the olefin metathesis step will depend upon the olefin metathesis catalyst used. In one embodiment, the temperature in the reaction zone will be within the range of from about 400° F. to about 1000° F., with temperatures in the range of from about 500° F. to about 850° F. usually being preferred. In general, the conversion of the olefins by olefin metathesis increases with an increase in pressure. Therefore, the selection of the optimal pressure for carrying out the process will usually be at the highest practical pressure under the circumstances. Accordingly, the pressure in the reaction zone should be maintained above 100 psig, and preferably the pressure should be maintained above 500 psig. The maximum practical pressure for the practice of the invention is about 5000 psig. More typically, the practical operating pressure will below about 3000 psig. The feedstock to the olefin metathesis reactor should contain a minimum of olefins, and preferably should contain no added hydrogen.

Tungsten catalysts are particularly preferred for carrying out the molecular averaging step, because the molecular averaging reaction will proceed under relatively mild conditions. When using the tungsten catalysts, the temperature should be maintained within the range of from about 400° F. (200° C.) to about 1000° F. (540° C.), with temperatures above about 500° F. (260° C.) and below about 800° F. being particularly desirable.

The olefin metathesis reaction described above is reversible, which means that the reaction proceeds toward a roughly thermodynamic equilibrium limit. Therefore, since the feed to the olefin metathesis zone has two streams of olefins at different molecular weights (i.e., ethylene and 2-butylenes), equilibrium will drive the reaction to produce a product stream having a molecular weight between that of the two streams (i.e., propylene). The zone in which the olefin metathesis occurs is referred to herein as an olefin metathesis zone. It is desirable to reduce the concentration of the desired products in the olefin metathesis zone to as low a concentration as possible to favor the reactions in the desired direction. As such, some routine experimentation may be necessary to find the optimal conditions for conducting the process.

In the event the catalyst deactivates with the time-on-stream, specific processes that are well known to those skilled in art are available for the regeneration of the catalysts.

Any number of reactors can be used, such as fixed bed, fluidized bed, ebulated bed, and the like. An example of a suitable reactor is a catalytic distillation reactor.

The dehydrogenation catalyst and the olefin metathesis catalyst can be, and typically are, present in separate reactors. However, for olefin metathesis catalysts which can tolerate the presence of the hydrogen generated in the dehydrogenation step, it may be possible to perform both steps in a single reactor. In a reactor having a layered fixed catalyst bed, the two components may, in such an embodiment, be separated in different layers within the bed.

The following language relates more specifically to the Meta-4 olefin metathesis conditions. In general, in the metathesis zone, the reagents (where ethylene can be used in excess, and the 2-butylene feed can be obtained from the previous step) pass over a bed of catalyst on which the metathesis reaction takes place, this generally being carried out continuously and generally comprising a reaction and a regeneration phase.

Then, for example, a metathesis catalyst can be used which contains rhenium oxide on alumina or on a compound derived from alumina such as a silica-alumina or boron oxide-alumina.

The reaction can then be carried out at a temperature of 0° C. to 100° C., preferably 25° C. to 75° C. It is preferably carried out in the liquid phase and then uses at least one reactor with a moving bed of catalyst, the feed moving from top to bottom or from bottom to top. When operating in the liquid phase, the HSV is preferably in the range 0.1 h⁻¹ to 10 h⁻¹, at a pressure which is preferably in the range 2 to 8 MPa.

The catalyst can then be employed in a moving bed and passes from a zone in which metathesis reaction conditions prevail and from which it can be withdrawn (for example from the bottom) to be sent to a zone in which regeneration conditions prevail, then returned to the reaction zone (for example to the head thereof).

Regarding regeneration, the catalyst withdrawn from the reaction zone is oxidized outside said regeneration zone with a gas containing 1% to 5% of oxygen at a first temperature, for example 350° C. to 750° C., then with a gas containing 5% to 40% of oxygen at a second temperature, for example 400° C. to 800° C., the second temperature being at least 50° C. above the first. The catalyst is then heated to the metathesis temperature and recycled to the reaction zone for re-use. This has been described in FR-B-2 608 595, for example.

In a further mode in which the metathesis section is employed, the metathesis catalyst can be disposed in a fixed bed in one or more reactors and each reactor is employed in succession in the reaction phase and in the regeneration phase, as described, for example, in FR-B-2 772 022.

According to FR-B-2 726 487 and FR-B-2 726 563, it is also possible to use a catalytic composition containing at least one compound of rhenium and aluminum with general formula:

O₃Re—O—(Al(OR)(L)ₓ—O)ₙ—ReO₃

(A) in which R represents a hydrocarbyl radical containing 1 to 40 carbon atoms; n is a number from 1 to 10; x is 0 or 1 and L represents the synthesis solvent.

The compositions containing the compound (A) can be used directly in a homogeneous phase, in the solvent resulting from their preparation, in the metathesis reaction. The compound (A) is obtained by contact of Re₂O₇ with a compound with general formula (RO)_{q}AlR′ᵣ, in which R is a hydrocarbon group containing 1 to 40 carbon atoms, R′ is a hydrocarbon radical containing 1 to 20 carbon atoms (preferably 1 to 6 carbon atoms); q and r are each one of the values 1 and 2, in which q+r32 3. The compound (RO)_{q}AlR′ᵣ is readily obtainable from trihydrocarbylaluminum AlR′₃ and the alcohol ROH. The compounds (A) are soluble in hydrocarbons, and normally do not require a co-catalyst.

The catalysts can be supported, the support possibly being organic or inorganic as described, for example, in FR-B-2 726 488.

More particularly, as described in FR-B-2 740 056, for example, a catalyst which contains at least one porous inorganic support, from 0.01% to 20% by weight of rhenium in the form of an oxide and 0.01% to 10% by weight of aluminum derived from an aluminum compound with formula (RO)_{q}AlR′ᵣ in which R is an alkyl group containing 1 to 40 carbon atoms, R′ is an alkyl group containing 1 to 20 carbon atoms, q and r are each one of the values 1 and 2 and q+r=3. That catalyst can be prepared by a process which comprises: (a) preparing a calcined precursor of the catalyst containing at least one porous inorganic support and rhenium; (b) bringing the precursor into contact with a compound with formula (RO)_{q}AIR′ᵣ obtained by reaction between ROH and AlR′₃; and (c) drying the resulting product.

It is also possible to use a catalyst containing rhenium, cesium and delta alumina as described, for example, in FR-B-2 784 040. The catalyst contains at least one rhenium compound, for example, deposited on a support principally composed of alumina; it is treated at a temperature of more than 750° C. and modified with at least one cesium compound.

Metathesis catalysts on a porous mineral support containing at least one rhenium, molybdenum or tungsten compound may also comprise silicon as described, for example, in FR-B-2 826 880.

In one embodiment, when using a supported catalyst, the support can more particularly consist of an alumina in the form of beads obtained using the “oil-drop” method, for example.

Metathesis is generally carried out in the gas phase at a temperature of 0° C. to 100° C., more particularly at 30° C. to 60° C., and still more particularly at a temperature of about 35° C.

As an example, the pressure can be 2 to 6 MPa, more particularly about 4.5 MPa. The mass flow rate with respect to the mass of catalyst of the combined feeds (butylenes and ethylene) at the reactor inlet can, for example, be 1 to 2 h⁻¹, more particularly about 1.5 h⁻¹.

In one embodiment, the metathesis zone also comprises a de-ethenizer to separate excess ethylene which is recycled at least in part to the metathesis zone, and a propylene separation column which can separate the high purity propylene which is produced, and unreacted butylenes. They are recycled in part to the metathesis reactor.

### Refractory Materials

In most cases, the metals in the catalyst mass (dehydrogenation and olefin metathesis) will be supported on a refractory material. Refractory materials suitable for use as a support for the metals include conventional refractory materials used in the manufacture of catalysts for use in the refining industry. Such materials include, but are not necessarily limited to, alumina, zirconia, silica, boria, magnesia, titania and other refractory oxide material or mixtures of two or more of any of the materials. The support may be a naturally occurring material, such as clay, or synthetic materials, such as silica-alumina and borosilicates. Molecular sieves, such as zeolites, also have been used as supports for the metals used in carrying out the dual functions of the catalyst mass. See, for example, U.S. Pat. No. 3,668,268. Mesoporous materials such as MCM-41 and MCM48, such as described in Kresge, C. T., et al., Nature (Vol. 359) pp. 710-712, 1992, may also be used as a refractory support. Other known refractory supports, such as carbon, may also serve as a support for the active form of the metals in certain embodiments. The support is preferably non-acidic, i.e., having few or no free acid sites on the molecule. Free acid sites on the support may be neutralized by means of alkali metal salts, such as those of lithium. Alumina, particularly alumina on which the acid sites have been neutralized by an alkali salt, such as lithium nitrate, is usually preferred as a support for the dehydrogenation/hydrogenation component, and silica is usually preferred as the support for the metathesis component. The preferred catalyst/support for the dehydrogenation step is Pt/silicalite, as this combination is believed to show the best resistance to fouling.

The amount of active metal present on the support may vary, but it must be at least a catalytically active amount, i.e., a sufficient amount to catalyze the desired reaction. In the case of the dehydrogenation/hydrogenation component, the active metal content will usually fall within the range from about 0.01 weight percent to about 50 weight percent on an elemental basis, with the range of from about 0.1 weight percent to about 20 weight percent being preferred. For the olefin metathesis component, the active metals content will usually fall within the range of from about 0.01 weight percent to about 50 weight percent on an elemental basis, with the range of from about 0.1 weight percent to about 25 weight percent being preferred.

### Separate Dehydrogenation of the C₂₋₄ Paraffin Fraction

In one embodiment, all or part of any C₂₋₄ paraffins formed during the methanol-to-olefins chemistry, or components thereof (i.e., ethane and/or butane) can be dehydrogenated to mono-olefins, separated into their component olefinic fractions, and, optionally, subjected to the olefin metathesis conditions described herein, ideally after converting any 1-butylene to 2-butylenes.

All or part of the hydrogen thus produced can be recycled into the process, for example, to increase the hydrogen/carbon monoxide ratio in the syngas. A well known dehydrogenation process is the UOP Pacol™ process. Syntroleum has demonstrated the feasibility of dehydrogenation of paraffins to mono-olefins. Thus, suitable dehydrogenation processes are well known and need not be described in more detail herein.

VI. Blends of The Olefins Described Herein with Conventionally-Derived Olefins

The olefins described herein can be blended with olefins derived from conventional sources, and subjected to polymerization conditions to form polyolefins, such as polyethylene, polypropylene, polybutylene, and co-polymers and blends thereof. In this embodiment, at least a portion of the olefins are derived from renewable resources.

### VII. Polymerization Conditions

Polyolefins, such as polyethylene and polypropylene, are produced through polymerization, which requires unique catalysts designed for each type of plastic. The olefins can be polymerized using conventional polymerization conditions, including conditions for producing low molecular weight and high molecular weight polyolefins. However, the possible presence of certain impurities, such as carbon monoxide, may require judicious selection of catalysts.

There are several methods for producing multimodal or broad molecular weight distribution resins: melt blending, reactor in series configuration, or single reactor with dual site catalysts. Use of a dual site catalyst for the production of a bimodal resin in a single reactor is also known.

Chromium catalysts for use in polyolefin production tend to broaden the molecular weight distribution and can in some cases produce bimodal molecular weight distribution. However, the low molecular part of these resins typically contains a substantial amount of the co-monomer. While a broadened molecular weight distribution provides acceptable processing properties, a bimodal molecular weight distribution can provide excellent properties.

Ziegler-Natta catalysts are capable of producing bimodal polyethylene using two reactors in series. Typically, in a first reactor, a low molecular weight homopolymer is formed by reaction between hydrogen and ethylene in the presence of the Ziegler-Natta catalyst. It is essential that excess hydrogen be used in this process and, as a result, it is necessary to remove all the hydrogen from the first reactor before the products are passed to the second reactor. In the second reactor, a copolymer of ethylene and hexene (which can be isolated from the Fischer-Tropsch synthesis described herein, and thus also obtainable from renewable resources) is made so as to produce a high molecular weight polyethylene.

Metallocene catalysts are also known in the production of polyolefins. For example, EP-A-0619325 describes a process for preparing polyolefins such as polyethylenes having a multimodal or at least bimodal molecular weight distribution. In this process, a catalyst system which includes at least two metallocenes is employed. The metallocenes used are, for example, a bis(cyclopentadienyl) zirconium dichloride and an ethylene-bis(indenyl) zirconium dichloride. By using the two different metallocene catalysts in the same reactor, a molecular weight distribution is obtained, which is at least bimodal.

U.S. Pat. No. 5,405,901 discloses the production of polyethylene blends in gas phase using two reactors for the production of films. A low density resin is produced in the first reactor and a high density resin is produced in the second reactor.

U.S. Pat. No. 5,284,613 discloses the production of bimodal molecular weight polyethylene resins containing two fractions of different molecular weight for the production of blown films exhibiting improved machine direction/transverse direction tear balance.

EP-A-0533154 discloses the production of ethylene polymer blends of a virgin or recycled low molecular weight ethylene polymer produced from a chromium-based catalyst and a high molecular weight ethylene polymer produced from a titanium-based catalyst.

U.S. Pat. No. 4,547,551 discloses the production of ethylene polymer blends of high molecular weight and low molecular weight ethylene polymers.

These and other catalysts and conditions for forming polyolefins are well known to those of skill in the art.

### VIII. Use of the Olefins, Polymers, and Other Products

The propylene produced as described herein can be used to produce polyolefins, or in any other conventional use for propylene. The polyolefins can be used in a variety of applications.

### Uses for Polyethylene/Polypropylene

Plastic automobile parts, bottles, containers, bags, industrial pipes, carpeting and toys are just some of the growing number of uses for polyolefins.

Polyethylenes which have high molecular weight generally have improved mechanical properties over their lower molecular weight counterparts. However, high molecular weight polyolefins can be difficult to process and can be costly to produce. Polyolefins having a multimodal molecular weight distribution (MWD) are desirable because they can combine the advantageous mechanical properties of high molecular weight fraction with the improved processing properties of one or more lower molecular weight fractions.

For many high density polyethylene (HDPE) applications, polyethylene with enhanced toughness, strength and environmental stress cracking resistance (ESCR) is important. These enhanced properties are more readily attainable with high molecular weight (HMW) polyethylene. However, as the molecular weight of the polymer increases, the processibility of the resin decreases. By providing a polymer with a broad or bimodal MWD, the desired properties that are characteristic of high molecular weight resin are retained while processibility, particularly extrudibility, can be improved.

Polyethylene resins are known for the production of pipes. Pipe resins require high resistance against slow crack growth as well as resistance to rapid crack propagation yielding impact toughness.

Ethylene is also often combined with other monomers to form co-polymers. Examples of these include polyethylene terephthalate, ethylene vinyl acetate, ethylene acrylic acid, and ethylene-propylene co-polymers. These are often used in asphalt, thermoadhesive films, and hotmelt adhesives, in the automotive, woodworking, textile, packaging, building and construction industries.

Polypropylene is a plastic polymer used in industry and in consumer goods as a structural plastic and as a fiber. Polypropylene is often used for dishwasher-safe food containers. The melting point of polypropylene is very high compared to many other plastics, at 320° F. (160° C.), so the hot water in the dishwasher will not warp the polypropylene dishware. Polyethylene can also be used to prepare containers, but it has a much lower melting point.

Polypropylene can be easily dyed, and is often used as a fiber in rugged and durable carpeting, for example, around swimming pools or paving miniature golf courses. Unlike nylon, polypropylene doesn't soak up water, making it ideal for uses where it will be constantly subject to moisture.

Polypropylene is also used in fabrics, upholstery, fibers, ropes, coatings, such as those for wires, automotive storage battery cases, automotive bumpers and accessories, refrigerator liners, under-ground storage tanks, industrial pallets, medical instruments, release liners for paper, film and foil.

The propylene can also be used combined with furan, which can be produced from corn, to produce 2-(2-propylene)furan (or 2-methyl vinylidene furan). This polymer can be used, for example, as a binder for fiberglass.

Polybutylene can be used as a hot-melt adhesive, as a binder, and as a co-polymer for a variety of commercial polymers, such as polybutylene terephthalate, polybutylene succinic anhydride (former by co-polymerizing butylene and maleic acid), which have a variety of uses.

Polypropylene can be functionalized (with compatibilizers) and blended with other polymers (polyethylene, polybutylene, nylon, EPDM, etc.)

All patents and publications disclosed herein are hereby incorporated by reference in their entirety and for all purposes. Modifications and variations of the present invention will be obvious to those skilled in the art from the foregoing detailed description of the invention.

1. A process for producing propylene, comprising the steps of:
a) obtaining a source of methanol, and converting the methanol to a product mixture comprising ethylene and propylene using methanol-to-olefins chemistry,
b) separating the ethylene from the propylene,
c) dimerizing a first portion of the ethylene to form a product mixture comprising 1-butylene,
d) isomerizing 1-butylene to form cis and trans 2-butylene,
e) subjecting a second portion of the ethylene and a first portion of the cis and trans 2-butylene to olefin metathesis to form a product mixture comprising propylene, and
f) isolating propylene.

2. The process of claim 1, wherein ethylene and cis and trans 2-butylene remaining from step f) are recycled to the olefin metathesis step.

3. The process of claim 1, wherein the methanol is prepared by converting syngas to methanol.

4. The process of claim 3, wherein the syngas is formed from a bio-based feedstock.

5. The process of claim 1, wherein any paraffins that are produced during the methanol-to-olefins step, or a portion thereof, are subjected to dehydrogenation conditions to form additional olefins.

6. The process of claim 1, further comprising polymerizing the propylene to form polypropylene.

7. The process of claim 1, further comprising copolymerizing the propylene with another monomer to form a polypropylene copolymer.

8. The process of claim 7, wherein the other monomer is selected from the group consisting of ethylene, butylene, maleic acid, and furan.

9. The process of claim 6, wherein the polymerization is performed using a Ziegler-Natta catalyst.

10. The process of claim 6, wherein the polymerization is performed using a metallocene catalyst.

11. A process for producing polypropylene or co-polymers thereof from renewable resources, comprising the steps of:
a) converting one or more renewable feedstocks capable of being converted to syngas into syngas,
b) converting the syngas to methanol,
c) converting the methanol to a product mixture comprising ethylene and propylene using methanol-to-olefins chemistry,
d) separating the ethylene from the propylene,
e) dimerizing a first portion of the ethylene to form a product mixture comprising 1-butylene,
f) isomerizing 1-butylene to form cis and trans 2-butylene,
g) subjecting a second portion of the ethylene and a first portion of the cis and trans 2-butylene to olefin metathesis to form a product mixture comprising propylene,
g) isolating propylene, and
h) subjecting the propylene, optionally in the presence of an additional monomer, to polymerization conditions.

12. The process of claim 11, wherein the polymer formed during the polymerization step is polypropylene, or a polyethylene-co-propylene co-polymer.

13. The process of claim 11, wherein the renewable resource is selected from the group consisting of glycerol, methanol, lignin, black liquor, switchgrass, bagasse, corn stover, rice hulls, sawdust, wood, hemicellulose, and algae.

14. The process of claim 11, wherein the other monomer is selected from the group consisting of ethylene, butylene, maleic acid, and furan.

15. The process of claim 11, wherein the polymerization is performed using a Ziegler-Natta catalyst.

16. The process of claim 11, wherein the polymerization is performed using a metallocene catalyst.

17. Polypropylene or copolymers thereof produced from a propylene feedstock comprising propylene produced by the process of claim 1, wherein all or a portion of the polypropylene can be identified by C14 dating as having been produced from a renewable source of propylene.

18. Polypropylene or copolymers thereof produced from a propylene feedstock comprising propylene produced by the process of claim 11, wherein all or a portion of the polypropylene can be identified by C14 dating as having been produced from a renewable source of propylene.
